(19) United States
(12) Patent Application Publication
(10) Pub. No.: US 2025/0072817 A1
(43) Pub. Date: Mar. 6, 2025

(54) Brain Health Assessment Battery

(71) Applicant: University of Rochester, Rochester, NY (US)

(72) Inventors: Kian Merchant-Borna, Rochester, NY (US); Jeffery J. Bazarian, Rochester, NY (US)

(21) Appl. No.: 18/821,182

(22) Filed: Aug. 30, 2024

Related U.S. Application Data
(60) Provisional application No. 63/579,984, filed on Sep. 1, 2023.

(51) Int. Cl. A61B 5/00; A61B 5/16

(52) U.S. Cl. CPC A61B 5/4064; A61B 5/162; A61B 5/7275

(57) ABSTRACT

Provided are methods of detecting concussion-level, neurologic dysfunction in a subject at risk of having developed concussion-level, neurologic dysfunction following exposure to repetitive, non-concussive head insults. Additionally provided are methods for preventing concussion-level, neurologic dysfunction in a subject at risk of developing such neurologic dysfunction.

### CROSS-REFERENCE TO RELATED APPLICATIONS

This application claims the benefit of the U.S. Provisional Application No. 63/579,984, filed Sep. 1, 2023, the disclosure of which is incorporated herein by reference in its entirety.

### STATEMENT OF GOVERNMENT SUPPORT

This invention was made with government support under W81XWH-14-2-0151 awarded by the Defense Health Agency, Medical Research and Development Branch. The government has certain rights in the invention.

### BACKGROUND OF THE INVENTION

Approximately 1.6 to 3.8 million athletes suffer a sports-related concussion (SRC) each year in the US, but virtually every contact sport athlete experiences repetitive head impacts (RHIs) which do not result in concussive symptoms such as loss of consciousness, amnesia, confusion, and headache (Bailes, J. E., et al., 2013, Journal of Neurosurgery, 119(5):1235-1245; McCrory, P., et al., 2017, British Journal of Sports Medicine, 51(11):838-847). These RHIs, commonly referred to as subconcussive head impacts, range in number from 175-1,444 per athlete in a single high school or collegiate football season (Greenwald, R. M., et al., 2008, Neurosurgery, 62(4):789-798; Crisco, J. J., et al., 2010, Journal of Athletic Training, 45(6):549-559; Broglio, S. P., et al., 2010, Medicine & Science in Sports & Exercise, 42(11):2064-2071; Crisco, J. J., et al., 2011, Journal of Biomechanics, 44(15):2673-2678; Montenigro, Ph. H., et al., 2016, Journal of Neurotrauma, 42(2):328-340). Among soccer players, the average number of purposeful headers per season ranges from 400-800, with some players exceeding 15,000 headers over the course of their career (Spiotta, A. M., et al., 2012, Neurosurgery, 70(1):11; Lipton, M. L., et al., 2013, Radiology, 268(3):850-857; Bailes, J. E., et al., 2013, Journal of Neurosurgery, 119(5):1235-1245; Matser, E. J., et al., 1999, Jama, 282(10):971-973; Matser, J., et al., 1998, Neurology, 51(3):791-796). While it is well recognized that concussions result in neurologic abnormalities such as cognitive deficits and postural instability, the neurologic manifestations of RHIs may be less well-known. Multiple studies, however, report concussion-level abnormalities in cognition (reaction time, attention, memory), vestibular function (postural stability, gait), and oculomotor function (convergence, saccades, smooth pursuits) (Stephen, S. J., et al., 2022, Journal of Head Trauma and Rehabilitation, 37(5):318-325). The frequency with which RHI-related neurologic dysfunction occurs has not been established but may be up to 20% of athlete-exposures (Kawata, K. T., et al., 2016, International Journal of Sports Medicine, 37:405-410).

Current SRC management guidelines recommend monitoring cognition and postural stability until they have returned to baseline before returning an athlete back to play (McCrory, P., et al., 2017, British Journal of Sports Medicine, 51(11):838-847). Conversely, there are no guidelines for monitoring the neurologic effects of RHIs. RHI-related disruptions in neurologic functions have the potential to reduce athletic performance and increase the risk of SRC as well as sports-related orthopedic and extremity injuries (Reams, N., et al., 2017, International Journal of Sports Physiology and Performance, 12(8):1100-1104; Hardy, R., et al., 2017, Physical Educator, 74(1):150-163; Wasserman, E. B., et al., 2015, American Journal of Sports Medicine, 43(5):1127-1133; Beckwith, J. G., et al., 2013, Medicine & Science in Sports & Exercise, 45(4):737-746; Nordstrom, A., et al., 2014, British Journal of Sports Medicine, 48(19):1447-1450; Brooks, M., et al., 2016, American Journal of Sports Medicine, 44(3):742-747).

Early detection of asymptomatic neurologic effects of RHIs could facilitate efforts to mitigate these risks. As such, there is need for a non-invasive method of quickly and accurately determining the risk of neurological damage over prolonged periods of exposure.

### SUMMARY OF THE INVENTION

In one aspect, the present disclosure provides methods of detecting neurologic dysfunction in a subject following exposure to repetitive, non-concussive head insults comprising: a) administering to the subject a series of tests to establish a baseline; b) administering to the subject, on a regular basis, the same series of tests; and c) monitoring the change between the results of the baseline tests and the results of the regularly administered tests.

In some embodiments, the series of tests comprises a King-Devick (K-D) test and Choice Reaction Time (CRT) test.

In some embodiments, repeated administration of tests in step b) occurs on a weekly basis. In some embodiments, step b) comprises administering the same series of tests after any activity in which a head insult has occurred.

In one aspect, the disclosure provides methods of preventing neurologic dysfunction in a subject following exposure to repetitive, non-concussive head insults comprising: a) administering to the subject a series of baseline tests; b) administering to the subject, on a regular basis, the same series of tests; c) monitoring the change between the results of the baseline tests and the results of the regularly administered tests; d) determining that the subject is at risk of concussion-level, neurologic dysfunction when the differences between the regular tests and the baseline tests trends upwards; e) altering the subject's behavior to reduce the influence of subsequent head insults on continued neurologic dysfunction; f) administering to the subject, on a regular basis, the same series of tests; and g) determining that the subject may resume normal activities when the test results are about the same as the results of the baseline test.

In some embodiments, the series of baseline tests comprises a King-Devick (KD) test and Choice Reaction Time (CRT) test.

In some embodiments, the repeated administration of tests in step b) occurs on a weekly basis. In some embodiments, step b) comprises administering the same series of tests after any activity in which a head insult has occurred.

In some embodiments, the repeated administration of tests in step f) occurs on a weekly basis. In some embodiments, the repeated administration of tests in step f) occurs on a biweekly basis.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 depicts representative histograms of changes from baseline in variables derived from the tests for non-concussed and concussed athletes. For non-concussed athletes, the differences are between the baseline measurements at year 2 and year 1. For concussed athletes, the differences are between the 24-hour post-injury and their baseline measurements. Each histogram was normalized to a unit area, generating population probability density functions. ΔK: King-Devick time (milliseconds); ΔK′: King-Devick errors (number); ΔI: Choice Reaction Time (ms).

FIG. 2A through FIG. 2C depict representative probability functions for the prediction of concussion risk based on changes in K-D time, K-D errors, and Choice RT. FIG. 2A depicts representative changes in probability score (from logistic regression) based on changes in K-D time of −2.5 seconds, 0 seconds, and +2.5 seconds. FIG. 2B depicts representative changes in LR score based on changes in K-D errors of two fewer errors, no change in errors, and 2 additional errors. FIG. 2C depicts representative changes in LR score based on changes in Choice RT of −1 ms, 0 ms, and +1 ms. Various lines represent different levels of errors in K-D time. Bottom to top: −5 seconds; −2.5 seconds; 0 seconds; +2.5 seconds; +5 seconds.

### DETAILED DESCRIPTION OF THE INVENTION

In one aspect, the disclosure is directed to methods for determining neurologic dysfunction following exposure to repetitive, non-concussive head insults, for example, after a subject has been subjected to a head insult. In some embodiments, the method comprises administering a series of tests to the subject to form a baseline and re-testing the subject to monitor for change in the test results. In some embodiments, the tests performed are a King-Devick test and a Choice Reaction Time test. In one embodiment, the method further comprises treating a subject determined to have neurologic disfunction.

In another aspect, the disclosure is related to methods of preventing concussion-level, neurologic dysfunction following exposure to repetitive, non-concussive head insults, for example, a subject involved in contact sports, manual labor, military activity, etc. In some embodiments, the method comprises administering series of tests to the subject to form a baseline, re-testing the subject periodically to monitor for change in the test results, instructing the subject to avoid activities with risk of head insult, re-testing regularly, and determining that the subject can resume normal activity once the results have returned to baseline. In some embodiments, the tests comprising the battery are a King-Devick test and a Choice Reaction Time test.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “patient,” “subject,” or “individual” are used interchangeably herein.

As used herein, the term “treatment” or “treating” is defined as the application or administration of a therapeutic agent, i.e., a compound of the invention (alone or in combination with another pharmaceutical agent), or therapy (e.g., vestibular therapy, physical therapy, behavioral therapy, motor exercises, ocular exercises, or cognitive exercises) to a patient, or application or administration of a therapeutic agent to an isolated tissue or cell from a patient (e.g., for diagnosis or ex vivo applications), wherein the patient has a disease or disorder contemplated herein, a sign or symptom of a disease or disorder contemplated herein, or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics.

“Measuring” or “measurement,” or alternatively “detecting” or “detection,” means assessing the presence, absence, quantity, or amount (which can be an effective amount) of either a given substance within a sample, including the derivation of qualitative or quantitative concentration levels of such substances, or otherwise evaluating the values or categorization of the substance or the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, in part, on the discovery that concussion-level, neurologic dysfunction can be detected by administration of a short battery of tests. Accordingly, in some embodiments, the invention is directed towards methods of detecting neurologic dysfunction by administering a short battery of tests to a subject to establish a baseline result, regular repeated administering of the battery of tests or administering the battery of tests after exposure to repetitive head impacts or individual head insults and comparing the results to the baseline results.

In other embodiments, the invention is directed towards methods of preventing further neurologic dysfunction. In one embodiment, the method comprises administering a short battery of tests to a subject to establish a baseline result, regular repeated administering of the battery of tests or administering the battery of tests after a head insult, comparing the results with the baseline results, altering the behavior of the subject to reduce the influence of subsequent head insults on continued neurologic dysfunction when the differences between the results trend upwards, regular repeated administering of the battery of tests, and determining that the subject can resume normal activity once the test results are approximately the same as the baseline.

### Test Battery

In one aspect, the present disclosure relates to batteries of tests for determining neurologic dysfunction. In one embodiment, the test battery comprises one or more tests for objective measurements of neurologic function. In some embodiments, the objective measurements are not related to symptomology. In some embodiments, the measurements can be conducted without specialized equipment. In some embodiments, the tests are one or more selected from the group consisting of Orientation/Maddock's Questions (MQ), Standardized Assessment of Concussion (SAC), modified BESS (Balance Error Score System, mBESS), tandem gait (TG), coordination examination

(CE), King-Devick (K-D), Near Point Convergence (NPC), Choice Reaction Time (CRT), and full BESS (fBESS). In some embodiments, the tests are one or more selected from the group consisting of KD, NPC, CRT, and fBESS. In some embodiments, the test battery comprises a combination of tests selected from the group consisting of: K-D and NPC; K-D and CRT; K-D, NPC, and CRT; K-D, NPC, and fBESS; and K-D, NPC, CRT and fBESS.

In some embodiments, the tests yield one or more scores. Examples of scores provided include, but are not limited to, time to complete the tests (K-D, milliseconds), number of errors in the test (K-D, number), distance to convergence (NPC, cm), reaction time (CRT, ms), and score (fBESS, number).

In some embodiments, the scores from the one or more tests are combined to form a composite score. In some embodiments, the composite score is generated from the combination of two or more scores selected from the group consisting of K-D time, K-D errors, NPC distance, CRT time, and fBESS score. In some embodiments, the combination is selected from the group consisting of: K-D time, K-D errors, and NPC distance; K-D time, K-D errors, and CRT time; K-D time, K-D errors, NPC distance, and CRT time; K-D time, K-D errors, NPC distance, and fBESS score; K-D errors, K-D time, NPC distance, CRT time, and fBESS score; K-D errors, NPC distance, and CRT time; K-D errors, NPC distance, CRT time, and fBESS score; and NPC distance and CRT time. In some embodiments, the composite score is calculated according to a diagnostic model.

In some embodiments, the composite score represents the probability that a subject has concussion-level, neurologic dysfunction. In some embodiments, the probability is expressed as a value between 0 and 1, wherein 0 indicates no chance of concussion-level, neurologic dysfunction and wherein the likelihood of concussion-level, neurologic dysfunction increases as the value approaches 1. In some embodiments, the probability is calculated according to a diagnostic model.

In some embodiments, one or more of the tests in the test battery are administered on a device. In some embodiments, the device is a computer, tablet, phone, or other generally available electronic device. In some embodiments, the device is a specialized electronic device for the tests. In some embodiments, the device provides individual test scores, a composite test score, or a combination thereof.

In some embodiments, one or more of the tests are administered by a qualified individual through the use of various analog implements. Analog implements that may be useful include, but are not limited to, test cards, stop watches, rulers, tape measures, padded mats, cushions, pillows, balls, balance boards, balance trainers, protractors, and goniometers.

### Methods

In one aspect, the present disclosure relates to methods of detecting concussion-level, neurologic dysfunction in a subject exposed to repetitive, non-concussive head insults. In some embodiments, the method comprises the steps of administering to the subject a test battery of the present invention to establish a baseline, re-administering the test battery, and monitoring the difference between the results of the baseline and re-administered test.

In some embodiments, the test battery is re-administered on a regular basis. In some embodiments, the test battery is re-administered every day, every two days, every three days, every four days, every five days, every six days, every seven, every eight days, every nine days, every ten days, every 11 days, every 12 days, every 13 days, every 14 days, every 15 days, every 16 days, every 17 days, every 18 days, every 19 days, every 20 days, every 21 days, every 22 days, every 23 days, every 24 days, every 25 days, every 26 days, every 27 days, every 28 days, every 29 days, every 30 days, or every 31 days.

In some embodiments, the test battery is re-administered once a week, every two weeks, every three weeks, every four weeks, every five weeks, every six weeks, every seven weeks, every eight weeks, every nine weeks, or every ten weeks.

In some embodiments, the test battery is re-administered once a month, every two months, every three months, every four months, every five months, every six months, every seven months, every eight months, every nine months, every ten months, every 11 months, or every 12 months.

In some embodiments, the test battery is re-administered once a year, every two years, every three years, every four years, every five years, every six years, every seven years, every eight years, every nine years, or every ten years.

In some embodiments, the test battery is re-administered after the subject experiences a head-insult or is exposed to repetitive, non-concussive head insults.

In some embodiments, the subject is determined to have neurologic dysfunction following exposure to repetitive, non-concussive head insults when the test results are different from the baseline results. In some embodiments, the subject is determined to have concussion-level, neurologic dysfunction following exposure to repetitive, non-concussive head insults when the test results are different from the baseline results. In some embodiments, the subject is determined to have concussion-level, neurologic dysfunction following exposure to repetitive, non-concussive head insults when the composite re-tested test score is between about 0.01% and about 10,000% greater than the baseline score. In some embodiments, the subject is determined to have concussion-level, neurologic dysfunction when the composite re-tested test score is between about 0.1% and about 1,000% greater than the baseline score. In some embodiments, the subject is determined to have concussion-level, neurologic dysfunction when the composite re-tested test score is between about 1% and about 100% greater than the baseline score. In some embodiments, the subject is determined to have concussion-level, neurologic dysfunction the composite re-tested test score is at least about 0.01%, at least about 0.05%, at least about 0.1%, at least about 0.15%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 320%, at least about 340%, at least about 360%, at least about 380%, at least about 400%, at least about 420%, at least about 440%, at least about 460%, at least about 480%, at least about 500%, at least about 550%, at least about 600%, at least about 650%, at least about 700%, at least about 750%, at least about 800%, at least about 850%, at least about 900%, at least about 950%, at least about 1,000%, at least about 1,100%, at least about 1,200%, at least about 1,300%, at least about 1,400%, at least about 1,500%, at least about 1,600%, at least about 1,700%, at least about 1,800%, at least about 1,900%, at least about 2,000%, at least about 3,000%, at least about 4,000%, at least about 5,000%, at least about 6,000%, at least about 7,000%, at least about 8,000%, at least about 9,000%, or at least about 10,000% greater than the baseline test result.

In some embodiments, the subject is determined to have concussion-level, neurologic dysfunction following exposure to repetitive, non-concussive head insults when the composite re-tested test score is between about 0.01% and about 100% less than the baseline score. In some embodiments, the subject is determined to have concussion-level, neurologic dysfunction when the composite re-tested test score is between about 0.1% and about 50% less than the baseline score. In some embodiments, the subject is determined to have concussion-level, neurologic dysfunction when the composite re-tested test score is between about 1% and about 10% less than the baseline score. In some embodiments, the subject is determined to have concussion-level, neurologic dysfunction when the composite re-tested test score is at least about 0.01%, at least about 0.05%, at least about 0.1%, at least about 0.15%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% less than the baseline test result.

In some embodiments, the subject is determined to have concussion-level, neurologic dysfunction following exposure to repetitive, non-concussive head insults when the calculated probability value after re-test is between about 0.5 and about 0.99, between about 0.6 and about 0.99, between about 0.7 and about 0.99, between about 0.8 and about 0.99, between about 0.9 and about 0.99, or between about 0.95 and about 0.99. In some embodiments, the subject is determined to have concussion-level, neurologic dysfunction when the calculated probability value after re-test is at least about 0.5, at least about 0.55, at least about 0.6, at least about 0.625, at least about 0.65, at least about 0.675, at least about 0.7, at least about 0.725, at least about 0.75, at least about 0.775, at least about 0.8, at least about 0.81, at least about 0.82, at least about 0.83, at least about 0.84, at least about 0.85, at least about 0.86, at least about 0.87, at least about 0.88, at least about 0.89, at least about 0.9, at least about 0.91, at least about 0.92, at least about 0.93, at least about 0.94, at least about 0.95, at least about 0.96, at least about 0.97, at least about 0.98, or at least about 0.99.

In some embodiments, the subject is determined to have concussion-level, neurologic dysfunction following exposure to repetitive, non-concussive head insults when the calculated probability after re-test is between about 0.1% and about 1,000% greater than the calculated baseline probability. In some embodiments, the subject is determined to have concussion-level, neurologic dysfunction when the probability after re-test is between about 1% and about 100% greater than the baseline probability. In some embodiments, the subject is determined to have concussion-level, neurologic dysfunction when the probability after re-test is at least about 0.01%, at least about 0.05%, at least about 0.1%, at least about 0.15%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 320%, at least about 340%, at least about 360%, at least about 380%, at least about 400%, at least about 420%, at least about 440%, at least about 460%, at least about 480%, at least about 500%, at least about 550%, at least about 600%, at least about 650%, at least about 700%, at least about 750%, at least about 800%, at least about 850%, at least about 900%, at least about 950%, at least about 1,000%, at least about 1,100%, at least about 1,200%, at least about 1,300%, at least about 1,400%, at least about 1,500%, at least about 1,600%, at least about 1,700%, at least about 1,800%, at least about 1,900%, at least about 2,000%, at least about 3,000%, at least about 4,000%, at least about 5,000%, at least about 6,000%, at least about 7,000%, at least about 8,000%, at least about 9,000%, or at least about 10,000% greater than the baseline probability.

In some embodiments, the method further comprises a step of treating the subject determined to have neurologic dysfunction. Treatments employed in the present invention are similar to the current standard of care for treatment of concussions. These treatments include any regimens currently recognized in the field, experimental treatments, and treatments still undergoing preliminary evaluation. Exemplary treatments include, but are not limited to, prescribed rest, prescribed rest with limited mental activities, prescribed rest with limited intermittent cardiovascular exercise, hydration maintenance, physical therapy, behavioral therapy, vestibular therapy, ocular therapy, motor skill exercises, balance exercises, reflex exercises, and combinations thereof.

In another aspect, the present invention provides methods of preventing concussion-level, neurologic dysfunction in people exposed to repetitive, non-concussive head insults. In some embodiments, the method comprises the steps of administering to the subject a test battery of the present invention to establish a baseline; re-administering the test battery; monitoring the difference between the results of the baseline and re-administered test; determining that the subject is at risk of concussion-level, neurologic dysfunction in people exposed to repetitive, non-concussive head insults when the re-administered test results trend away from the baseline results; instructing the subject to avoid activities with a risk of head insult; re-administering the test battery; monitoring the differences between the results of the baseline and the re-administered test; and determining that the subject may resume normal activities when the re-administered test results are not significantly different from the baseline results.

In some embodiments, the test battery is re-administered on a regular basis. In some embodiments, the test battery is re-administered every day, every two days, every three days, every four days, every five days, every six days, every seven, every eight days, every nine days, every ten days, every 11 days, every 12 days, every 13days, every 14 days, every 15 days, every 16 days, every 17 days, every 18 days, every 19 days, every 20 days, every 21 days, every 22 days, every 23 days, every 24 days, every 25 days, every 26 days, every 27 days, every 28 days, every 29 days, every 30days, or every 31 days.

In some embodiments, the test battery is re-administered once a week, every two weeks, every three weeks, every four weeks, every five weeks, every six weeks, every seven weeks, every eight weeks, every nine weeks, or every ten weeks.

In some embodiments, the test battery is re-administered once a month, every two months, every three months, every four months, every five months, every six months, every seven months, every eight months, every nine months, every ten months, every 11 months, or every 12 months.

In some embodiments, the test battery is re-administered once a year, every two years, every three years, every four years, every five years, every six years, every seven years, every eight years, every nine years, or every ten years.

In some embodiments, the test battery is re-administered after the subject experiences a head-insult.

In some embodiments, the subject is determined to be at risk of neurologic dysfunction following exposure to repetitive, non-concussive head insults when the test results are different from the baseline results. In some embodiments, the subject is determined to be at risk of neurologic dysfunction when the composite re-tested test score is between about 0.01% and about 10,000% greater than the baseline score. In some embodiments, the subject is determined to be at risk of neurologic dysfunction when the composite re-tested test score is between about 0.1% and about 1,000% greater than the baseline score. In some embodiments, the subject is determined to have neurologic dysfunction when the composite re-tested test score is between about 1% and about 100% greater than the baseline score. In some embodiments, the subject is determined to have neurologic dysfunction the composite re-tested test score is at least about 0.01%, at least about 0.05%, at least about 0.1%, at least about 0.15%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 320%, at least about 340%, at least about 360%, at least about 380%, at least about 400%, at least about 420%, at least about 440%, at least about 460%, at least about 480%, at least about 500%, at least about 550%, at least about 600%, at least about 650%, at least about 700%, at least about 750%, at least about 800%, at least about 850%, at least about 900%, at least about 950%, at least about 1,000%, at least about 1,100%, at least about 1,200%, at least about 1,300%, at least about 1,400%, at least about 1,500%, at least about 1,600%, at least about 1,700%, at least about 1,800%, at least about 1,900%, at least about 2,000%, at least about 3,000%, at least about 4,000%, at least about 5,000%, at least about 6,000%, at least about 7,000%, at least about 8,000%, at least about 9,000%, or at least about 10,000% greater than the baseline test result.

In some embodiments, the subject is determined to be at risk of neurologic dysfunction following exposure to repetitive, non-concussive head insults when the composite re-tested test score is between about 0.01% and about 100% less than the baseline score. In some embodiments, the subject is determined to be at risk of neurologic dysfunction when the composite re-tested test score is between about 0.1% and about 50% less than the baseline score. In some embodiments, the subject is determined to be at risk of neurologic dysfunction when the composite re-tested test score is between about 1% and about 10% less than the baseline score. In some embodiments, the subject is determined to be at risk of neurologic dysfunction when the composite re-tested test score is at least about 0.01%, at least about 0.05%, at least about 0.1%, at least about 0.15%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% less than the baseline test result.

In some embodiments, the subject is determined to be at risk of neurologic dysfunction following exposure to repetitive, non-concussive head insults when the calculated probability value after re-test is between about 0.5 and about 0.99, between about 0.6 and about 0.99, between about 0.7 and about 0.99, between about 0.8 and about 0.99, between about 0.9 and about 0.99, or between about 0.95 and about 0.99. In some embodiments, the subject is determined to be at risk of neurologic dysfunction when the calculated probability value after re-test is at least about 0.5, at least about 0.55, at least about 0.6, at least about 0.625, at least about 0.65, at least about 0.675, at least about 0.7, at least about 0.725, at least about 0.75, at least about 0.775, at least about 0.8, at least about 0.81, at least about 0.82, at least about 0.83, at least about 0.84, at least about 0.85, at least about 0.86, at least about 0.87, at least about 0.88, at least about 0.89, at least about 0.9, at least about 0.91, at least about 0.92, at least about 0.93, at least about 0.94, at least about 0.95, at least about 0.96, at least about 0.97, at least about 0.98, or at least about 0.99.

In some embodiments, the subject is determined to be at risk of neurologic dysfunction following exposure to repetitive, non-concussive head insults when the calculated probability after re-test is between about 0.1% and about 1,000% greater than the calculated baseline probability. In some embodiments, the subject is determined to be at risk of neurologic dysfunction when the probability after re-test is between about 1% and about 100% greater than the baseline probability. In some embodiments, the subject is determined to be at risk of neurologic dysfunction when the probability after re-test is at least about 0.01%, at least about 0.05%, at least about 0.1%, at least about 0.15%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 320%, at least about 340%, at least about 360%, at least about 380%, at least about 400%, at least about 420%, at least about 440%, at least about 460%, at least about 480%, at least about 500%, at least about 550%, at least about 600%, at least about 650%, at least about 700%, at least about 750%, at least about 800%, at least about 850%, at least about 900%, at least about 950%, at least about 1,000%, at least about 1,100%, at least about 1,200%, at least about 1,300%, at least about 1,400%, at least about 1,500%, at least about 1,600%, at least about 1,700%, at least about 1,800%, at least about 1,900%, at least about 2,000%, at least about 3,000%, at least about 4,000%, at least about 5,000%, at least about 6,000%, at least about 7,000%, at least about 8,000%, at least about 9,000%, or at least about 10,000% greater than the baseline probability.

In some embodiments, the subject is permitted to resume normal activities when the re-administered composite score is not significantly different from the baseline composite score. In some embodiments, re-administered composite score is not significantly different from the baseline composite score when it is less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.45%, about 0.4%, about 0.35%, about 0.3%, about 0.25%, about 0.2%, about 0.15%, about 0.1%, 0.75%, about 0.05%, about 0.04%, about 0.03%, about 0.02%, or about 0.01% increased relative to the baseline composite score.

In some embodiments, re-administered composite score is not significantly different from the baseline composite score when it is less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.45%, about 0.4%, about 0.35%, about 0.3%, about 0.25%, about 0.2%, about 0.15%, about 0.1%, 0.75%, about 0.05%, about 0.04%, about 0.03%, about 0.02%, or about 0.01% decreased relative to the baseline composite score.

In some embodiments, the subject is permitted to resume normal activities when the re-administered probability is not significantly different from the baseline probability. In some embodiments, re-administered probability score is not significantly different from the probability when it is less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.45%, about 0.4%, about 0.35%, about 0.3%, about 0.25%, about 0.2%, about 0.15%, about 0.1%, 0.75%, about 0.05%, about 0.04%, about 0.03%, about 0.02%, or about 0.01% increased relative to the baseline probability.

### Computing Device

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written, in particular programming languages, or executed on particular operating systems or computing platforms, it is understood that the system and method of the present invention is not limited to any particular computing language, platform, or combination thereof. Software executing the algorithms described herein may be written in any programming language known in the art, compiled, or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

### EXPERIMENTAL EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Without further description, it is believed that one of ordinary skill in the art can, using the preceding description and the following illustrative examples, make and utilize the present invention and practice the claimed methods. The following working examples, therefore, point out specific embodiments of the present invention, and are not to be construed as limiting in any way the remainder of the disclosure.

### Example 1: Identification of a Test Battery for Detecting Concussion-Level, Neurologic Dysfunction

To develop a test battery able to accurately classify concussion-level, neurologic dysfunction in people exposed to repetitive, non-concussive head insults, existing, validated sideline concussion tests able to accurately detect concussion-level, neurologic dysfunction were identified using data archived by the NCAA CARE Consortium. Because the use of this test battery involves repeated, intermittent administration, selection of test variables was limited to those that are quick and easy to administer, and do not require special equipment such as force plates or eye trackers.

Moreover, given the low sensitivity and specificity of concussion symptoms, selection of tests was further limited to those that do not involve symptoms (Iverson, G. L., et al., 2015, JAMA Pediatrics, 169(12):1132-1140; Iverson, G. L., et al., 2003, Applied Neuropsychology, 10(3):137-144; Asken, B. M., et al., 2017, The Clinical Neuropsychologist, 31(1):138-153).

Machine-learning techniques were used to identify a combination of sideline tests and their cutoff scores that best separated or “classified” concussed athletes from non-concussed athletes (Jordan, m. I., et al., 2015, Science, 349(6245):255-260). This combination of tests and their cutoffs represented the novel test battery. To derive and validate this battery, archived sideline test data contained in the NCAA/DOD CARE Consortium were analyzed (Broglio, S. P., et al., 2017, Sports Medicine, 47(7):1437-1451; NCAA-DOD Grad Alliance Care Consortium, CARE Consortium Database).

The CARE Consortium database contains results from multiple sideline tests, some of which have been previously validated and are recommended by the 5th International Conference on Concussion in Sport (McCrory, P., et al., 2017, British Journal of Sports Medicine, 51(11):838-847). Sideline tests eligible for inclusion into the machine learning analysis were those collected at baselines, as well as at 24 hours after injury, were objective measures of neurologic function, were not symptom-based, and did not require special equipment such as force plates or eye trackers.

Nine candidate sideline tests collected and archived by the NCAA/DOD CARE consortium were initially identified for inclusion into the machine learning process (NCAA-DOD Grad Alliance Care Consortium, CARE Consortium Database): Orientation/Maddock's Questions score (SCAT), Standardized Assessment of Concussion score (SCAT), modified BESS (Balance Error Scoring System) score (SCAT), tandem gate score (SCAT), coordination exam result (SCAT), King-Devick time (seconds) and errors (number), Near Point Convergence (cm, VOMS), Choice Reaction (milliseconds, ImPACT, CNS, ANAM), and full BESS score.

Although numerous subjects enrolled in the CARE consortium underwent SCAT testing (Sport Concussion Assessment Tool), the non-symptom scores (Maddock's Questions, Standardized Assessment of Concussion, modified BESS, tandem gait, and coordination exam) were not entered into the CARE database as individual scores. As such, the remaining tests were King-Devick, Near Point Convergence, Choice Reaction, and full BESS score.

Administration of Tests: When a concussion was suspected, the athlete completed a measure of neurocognitive function, balance assessment, and symptom report within 6 hours of injury, 24-48 hours post-injury, when cleared to begin the return to play process, and when cleared for unrestricted return to play. The baseline assessment required 55-60 minutes to complete, while the 6-hour time point took approximately 20 minutes and assessments at the remaining time points took 35-40 minutes.

King-Devick Test: The K-D test, a rapid number naming test, involves reading aloud a series of single-digit numbers from left to right on three test cards. The K-D test includes one practice (demonstration) card and three test cards of increasing difficulty. The test was conducted using physical cards with hand-timing or via tablet in which the device times the administration. Participants read the numbers on each card from left to right as quickly as possible while trying not to make any errors. The test administrator recorded the time to complete the test and any errors. The sum of three test card time scores constituted the summary score for the entire test, the K-D time score. Numbers of errors made in reading the test cards were also recorded (Galetta, K. M., et al., 2011, Journal of the Neurological Sciences, 309(1-2):34-39; Galetta, K. M., et al., 2016, Concussion, 1(2):CNC8).

ImPACT Reaction Time: The ImPACT reaction time test yields a composite choice reaction time score calculated at the end of the ImPACT test from the choice reaction components of three ImPACT modules: X's and O's, Symbol Match, and Color Match. For example, the Color Match module measures impulse control/response inhibition. Word names were displayed on the screen in the same color as the word (e.g., RED), or in a different color (GREEN or BLUE), and the test taker was instructed to click in the box as quickly as possible only if the word name is presented in the matching color. In addition to providing a reaction time score, this task also provided an error score (ImPACT Applications, Inc., ImPACT Version 4 Administration and Interpretation Manual, 2021).

Near Point Convergence (NPC) from VOMS: The Near Point Convergence test measures the ability to view a near target without double vision. The participant was seated and wearing corrective lenses (if needed). The examiner, seated in front of the participant, observed the eye movements as the participant focused on a small target (approximately 14-point font size) at arm's length and slowly brought it toward the tip of the participant's nose. The participant was instructed to stop moving the target when s/he saw two distinct images or when the examiner observed an outward deviation of one eye. Blurring of the image was ignored. The distance in cm between target and the tip of nose was measured 3 times and averaged. The examiner recorded ratings of symptom exacerbation of headache, dizziness, nausea, and fogginess after the test. Abnormal NPC was defined as diplopia ≥6 cm from the tip of the nose (Mucha, A., et al., 2014, American Journal of Sports medicine, 42(10):2479-2486).

Balance Error Score System: The BESS is a postural stability measure that was administered in 5 min while the athlete completed three 20-second stance trials (i.e., double leg. single leg, tandem stance) on firm and foam surfaces (Broglio, S. P., eta l., 2018, Sports Medicine, 48(5):1255-1268). The number of errors was recorded (moving the hands off of the iliac crests, opening the eyes, step stumble or fall, abduction or flexion of the hip beyond 30°, lifting the forefoot or heel off of the testing surface, and/or remaining out of the proper testing position for greater than 5 seconds). The maximum total number of errors for any single condition is 10. If a participant committed multiple errors simultaneously, only one error was recorded. Participants unable to maintain the testing procedure for a minimum of 5 seconds were assigned the highest possible score, 10, for that testing condition. Each of the twenty-second trials was scored by summing participant errors and/or deviations from the proper stance.

Analysis of Scores: Subject-specific changes in these 4 sideline tests were analyzed, rather than evaluating just post-injury test performance. For concussed athletes, subject-specific changes were defined as the change in sideline test score from baseline to 24-hours post-injury. For non-concussed athletes (contact and non-contact), subject-specific change was defined as the change from baseline at year 1 to baseline at year 2. To be included in this analysis, concussed athletes needed to have at least 1 of the 4 sideline tests under consideration at both the baseline and 24-hour time points. Similarly, included non-concussed athletes (contact and non-contact) needed to have at least 1 of the 4 sideline tests under consideration at both the baseline year 1 and baseline year 2. Once subjects meeting these criteria were identified, they were split such that 80% of subjects in each group were used to derive or “train” the classifier, and the remaining 20% were used for validation.

In order to predict concussion risk and identify a cutoff score that best discriminated concussed athletes from non-concussed athletes, the following measures from the sideline tests were used, and their values denoted by the following variables:
K: King-Devick Baseline Attempt #1 Total Time
K′: King-Devick Baseline Attempt #1 Total Errors
V: VOMS Near-Point Convergence Measure #3
I: ImPACT Reaction Time Composite Score (Choice RT)
B: BESS Total Score

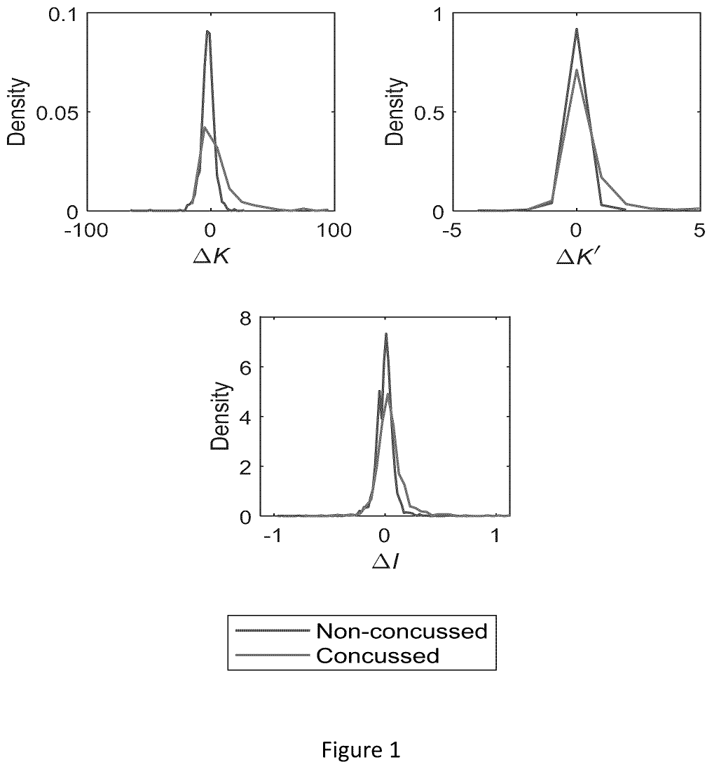

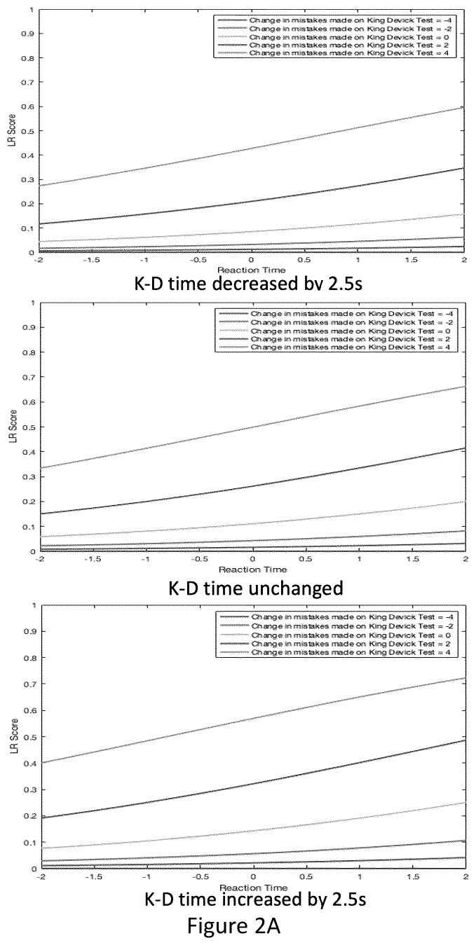

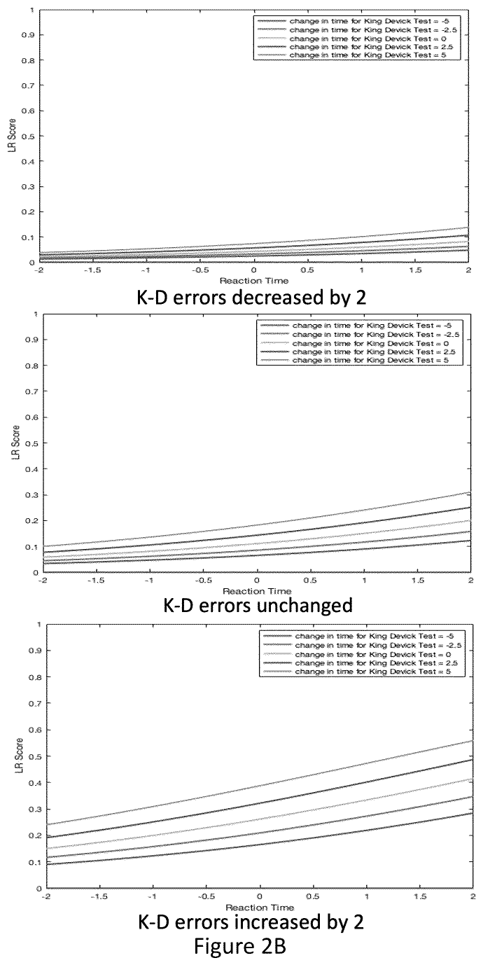

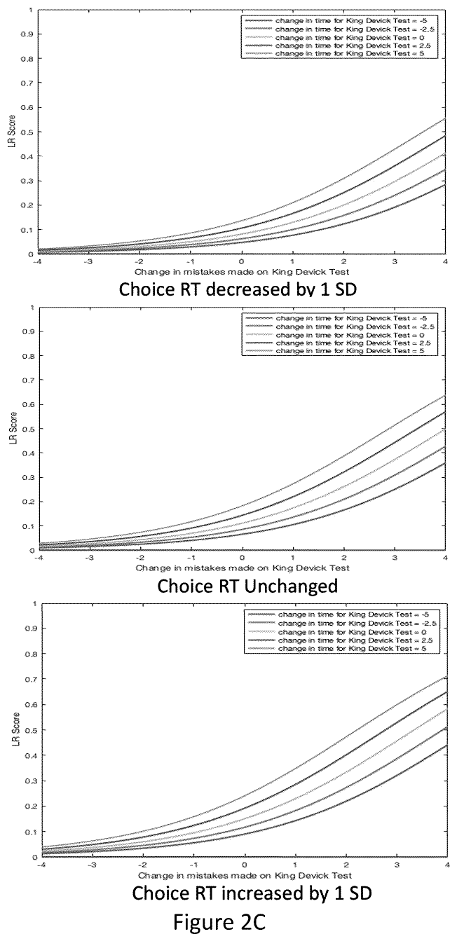

For each of these values, their change across two time points, which were denoted ΔK, ΔK′, etc. were examined. FIG. 1 illustrates estimated densities (histograms normalized to unit area) for changes in each variable for both non-concussed and concussed athletes. A breakdown of what data was available for different subjects is presented in Table 1. The origins of the data are presented in Table 2.

In order to derive the test battery, AUCs were computed for all possible combinations of these 5 tests using 4 different machine learning approaches: Logistic Regression (LR), Naïve Bayes (NB), discriminative, and tree based. The AUC values from the various approaches are presented in Table 3.

TABLE 1
Total number of non-concussed and concussed athletes
for various combinations of test changes.

| ΔK | ΔK′ | ΔV | ΔI | ΔB | Non-Concussed | Concussed |
|---|---|---|---|---|---|---|
| + | | | | | 1223 | 178 |
| | + | | | | 1226 | 178 |
| | | + | | | 338 | 185 |
| | | | + | | 4155 | 875 |
| | | | | + | 5372 | 1138 |
| + | + | | | | 1223 | 178 |
| + | | + | | | 121 | 57 |
| + | | | + | | 1193 | 160 |
| + | | | | + | 1124 | 138 |
| + | + | + | | | 121 | 57 |
| + | + | | + | | 1193 | 160 |
| + | + | | | + | 1124 | 138 |
| + | | + | + | | 120 | 57 |
| + | | + | | + | 121 | 55 |
| + | | | + | + | 1099 | 129 |
| + | + | + | + | | 120 | 57 |
| + | + | + | | + | 121 | 55 |
| + | + | | + | + | 1099 | 129 |
| + | | + | + | + | 120 | 55 |
| + | + | + | + | + | 120 | 55 |
| | + | + | | | 121 | 57 |
| | + | | + | | 1196 | 160 |
| | + | | | + | 1127 | 138 |
| | + | + | + | | 120 | 57 |
| | + | + | | + | 121 | 55 |
| | + | | + | + | 1102 | 129 |
| | + | + | + | + | 120 | 55 |
| | | + | + | | 329 | 176 |
| | | + | | + | 334 | 177 |
| | | + | + | + | 325 | 169 |
| | | | + | + | 3801 | 794 |

TABLE 2
Number of subjects given tests at each site data was derived from.

| | Non-Concussed | | | | Concussed | | | |
|---|---|---|---|---|---|---|---|---|
| Site | K-D | NPC | CRT | fBESS | K-D | NPC | CRT | fBESS |
| Azusa Pacific | 0 | 0 | 225 | 228 | 0 | 0 | 62 | 62 |
| Bloomsburg | 0 | 0 | 0 | 0 | 0 | 0 | 14 | 13 |
| Cal Lutheran | 0 | 0 | 165 | 172 | 0 | 0 | 32 | 32 |
| Humboldt State | 191 | 121 | 192 | 189 | 55 | 47 | 55 | 54 |
| Indiana U | 0 | 1 | 75 | 0 | 0 | 23 | 26 | 28 |
| Princeton | 0 | 0 | 297 | 251 | 0 | 0 | 39 | 38 |
| Temple U | 0 | 0 | 0 | 0 | 0 | 0 | 2 | 19 |
| U Chicago | 0 | 0 | 0 | 0 | 0 | 0 | 13 | 24 |
| U Delaware | 339 | 0 | 342 | 329 | 42 | 10 | 44 | 41 |
| U Florida | 332 | 0 | 339 | 341 | 22 | 0 | 19 | 24 |
| U Georgia | 1 | 0 | 0 | 284 | 1 | 0 | 0 | 34 |
| U Miami | 0 | 0 | 0 | 0 | 0 | 0 | 11 | 15 |
| U Michigan | 0 | 0 | 0 | 596 | 0 | 0 | 0 | 105 |
| U North Carolina | 0 | 0 | 0 | 173 | 0 | 1 | 1 | 50 |
| U North Georgia | 0 | 0 | 0 | 0 | 0 | 0 | 10 | 10 |
| U Oklahoma | 0 | 0 | 0 | 264 | 0 | 0 | 0 | 30 |
| U Penn | 0 | 0 | 0 | 0 | 1 | 0 | 6 | 19 |
| U Pittsburgh | 0 | 117 | 334 | 334 | 0 | 27 | 60 | 55 |
| U Rochester | 0 | 0 | 277 | 275 | 0 | 0 | 45 | 50 |
| U Washington | 360 | 0 | 364 | 290 | 57 | 0 | 54 | 24 |
| U Wisconsin | 0 | 99 | 421 | 422 | 0 | 36 | 80 | 85 |
| UCLA | 0 | 0 | 253 | 246 | 0 | 0 | 43 | 52 |
| US Air Force | 0 | 0 | 563 | 574 | 0 | 0 | 106 | 115 |
| US Coast Guard | 0 | 0 | 3 | 119 | 0 | 0 | 4 | 4 |
| US Military | 0 | 0 | 245 | 240 | 0 | 0 | 48 | 54 |
| Virginia Tech | 0 | 0 | 57 | 45 | 0 | 0 | 11 | 13 |
| Wake Forest U | 0 | 0 | 0 | 0 | 0 | 33 | 44 | 42 |
| Wilmington | 0 | 0 | 0 | 0 | 0 | 0 | 29 | 32 |
| Winston-Salem | 0 | 0 | 0 | 0 | 0 | 8 | 15 | 14 |
| Total | 1223 | 338 | 4152 | 5372 | 178 | 185 | 873 | 1138 |

TABLE 3
AUC values and lower 95% confidence intervals (Cis) for various
test combinations. Bolded items have AUC values ≥0.7000.

| ΔK | LR | NB | Disc. | Tree |
|---|---|---|---|---|
| ΔK, ΔI | 0.6829, 0.6327 | 0.6746, 0.6242 | 0.6847, 0.6346 | 0.5589, 0.5116 |
| ΔK, ΔB | 0.6488, 0.5916 | 0.6412, 0.5832 | 0.6489, 0.5912 | 0.6133, 0.5757 |
| ΔK, ΔK,′ ΔV | 0.7074, 0.6122 | 0.6826, 0.5853 | 0.7144, 0.6234 | 0.6837, 0.6140 |
| ΔK, ΔK′, ΔI | 0.7137, 0.6648 | 0.7243, 0.6741 | 0.7127, 0.6636 | 0.7188, 0.6766 |
| ΔK, ΔK′, ΔB | 0.6648, 0.6079 | 0.6736, 0.6149 | 0.6631, 0.6056 | 0.6595, 0.6164 |
| ΔK, ΔV, ΔI | 0.6461, 0.5480 | 0.6199, 0.5228 | 0.6553, 0.5585 | 0.6744, 0.6021 |
| ΔK, ΔV, ΔB | 0.6141, 0.5112 | 0.6121, 0.5133 | 0.6259, 0.5242 | 0.5797, 0.5231 |
| ΔK, ΔI, ΔB | 0.6678, 0.6100 | 0.6580, 0.6011 | 0.6699, 0.6115 | 0.6211, 0.5809 |
| ΔK, ΔK′, ΔV, ΔI | 0.7308, 0.6373 | 0.6867, 0.5937 | 0.7250, 0.6351 | 0.7520, 0.6789 |
| ΔK, ΔK′, ΔV, ΔB | 0.7120, 0.6141 | 0.6878, 0.5879 | 0.7107, 0.6170 | 0.6815, 0.6101 |
| ΔK, ΔK′, ΔI, ΔB | 0.6816, 0.6239 | 0.6847, 0.6257 | 0.6797, 0.6212 | 0.6522, 0.6048 |
| ΔK, ΔV, ΔI, ΔB | 0.6632, 0.5668 | 0.6582, 0.5646 | 0.6686, 0.5729 | 0.6848, 0.6124 |
| ΔK, ΔK′, ΔV, ΔI, ΔB | 0.7401, 0.6455 | 0.6930, 0.5992 | 0.7330, 0.6422 | 0.7527, 0.6779 |
| ΔK′, ΔV | 0.6707, 0.5732 | 0.6716, 0.5768 | 0.6799, 0.5846 | 0.6053, 0.5489 |
| ΔK′, ΔI | 0.6667, 0.6117 | 0.6137, 0.5575 | 0.6682, 0.6130 | 0.5000, 0.5000 |
| ΔK′, ΔB | 0.5693, 0.5038 | 0.6079, 0.5429 | 0.4777, 0.4149 | 0.5000, 0.5000 |
| ΔK′, ΔV, ΔI | 0.7265, 0.6320 | 0.7277, 0.6383 | 0.7248, 0.6331 | 0.7518, 0.6787 |
| ΔK′, ΔV, ΔB | 0.6742, 0.5761 | 0.6938, .05998 | 0.6754, 0.5816 | 0.5781, 0.4966 |
| ΔK′, ΔI, ΔB | 0.6323, 0.5662 | 0.4940, 0.4816 | 0.4741, 0.4090 | 0.5000, 0.5000 |
| ΔK′, ΔV, ΔI, ΔB | 0.7362, 0.6424 | 0.6843, 0.5904 | 0.7327, 0.6415 | 0.7534, 0.6787 |
| ΔV, ΔI | 0.6816, 0.6288 | 0.6882, 0.6371 | 0.6819, 0.6292 | 0.7113, 0.6655 |
| ΔV, ΔB | 0.6317, 0.5785 | 0.6374, 0.5845 | 0.6365, 0.5834 | 0.5185, 0.4681 |
| ΔV, ΔI, ΔB | 0.6874, 0.6330 | 0.6605, 0.6067 | 0.6866, 0.6323 | 0.6378, 0.5971 |
| ΔI, ΔB | 0.6198, 0.5943 | 0.6062, 0.5810 | 0.5666, 0.5419 | 0.5763, 0.6501 |

Of the testing combinations evaluated in Table 2, only two combinations yielded consistent AUC values greater than 0.7000 independent of the machine learning method that was implemented. Unexpectedly, the combination of all five test parameters was not among these two combinations, rather the combinations were of ΔK, ΔK′, and ΔI or ΔK′, ΔV, and ΔI. While these combinations yield similar results, the combination of ΔK, ΔK′, and ΔI requires only two tests, King-Devick and ImPACT Reaction Time 10 whereas the combination of ΔK′, ΔV, and ΔI requires both of these tests as well as the VOMS Near-Point Convergence test. Therefore, the combination of King-Devick and ImPACT Reaction Time was selected for further evaluation.

### Example 2: Evaluation of Testing

To investigate the feasibility of the testing battery with a King-Devick and Reaction Time test, a brief (estimated 5-7 mins) neurologic test battery (Collegiate Athlete Brief Test Battery) was administered to football and women's soccer players at the University of Rochester and University at Buffalo during the 2022 fall sport season. Forty-seven athletes participating in football (24) or women's soccer (23) were selected based on their enrollment status, active engagement in their sport, ability to provide written consent, willingness to participate in follow-up, and absence of a concussion ≤30 days prior to test enrollment.

Pre-season, subjects underwent a King-Devick test and a Choice Reaction Time test. Subjects were also asked for demographic information, including age, sex, race, ethnicity, sport/position, prescribed medications, history of concussions, history of attention deficit disorder/attention deficit hyperactivity disorder (ADD/ADHD), history of learning disabilities, and history of migraine headaches.

During the season, subjects underwent weekly King-Devick and Choice Reaction Time tests. For football players, the tests were administered weekly during routine med checks. For soccer players, the tests were administered once weekly before afternoon/evening practice. All tests were administered 12-36 hours after the last contact event the subject was exposed to. Weekly head impact exposure was estimated based on self-reported number of head impacts.

The disclosures of each and every patent, patent application, and publication cited herein are hereby incorporated herein by reference in their entirety. While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of determining neurologic dysfunction in a subject comprising:
a) administering to the subject a series of baseline tests;
b) administering to the subject, on a regular basis, the same series of tests; and
c) monitoring the change between the results of the baseline tests and the results of the regularly administered tests.

2. The method of claim 1, wherein the series of tests comprises a King-Devick (K-D) test and Choice Reaction Time (CRT) test.

3. The method of claim 1, wherein step b) comprises administering the series of tests on a weekly basis.

4. The method of claim 1, wherein step b) further comprises administering the series of tests after any activity in which a head insult has occurred.

5. A method of preventing neurologic dysfunction in a subject comprising
a) administering to the subject a series of baseline tests;
b) administering to the subject, on a regular basis, the same series of tests;
c) monitoring the change between the results of the baseline tests and the results of the regularly administered tests;
d) determining that the subject is at risk of concussion-level, neurologic dysfunction when the differences between the regular tests and the baseline test trend upwards;
e) altering the behavior of the subject to reduce the influence of subsequent head insults on continued neurologic dysfunction;
f) administering to the subject, on a regular basis, the same series of tests; and
g) determining that the subject may resume normal activities when the test results are about the same as the results of the baseline test.

6. The method of claim 5, wherein the series of tests comprises a K-D test and CRT test.

7. The method claim 5, wherein step b) comprises administering the series of tests on a weekly basis.

8. The method of claim 5, wherein step b) further comprises administering the series of tests after any activity in which a head insult has occurred.

9. The method of claim 5, wherein step f) comprises administering the series of tests on a weekly basis.

10. The method of any of claim 5, wherein step f) comprises administering the series of tests on a biweekly basis.
